SEQUENCE LISTING (4 sequences)

SEQ ID NO: 1 - protein, 35 aa, Adeno-associated virus 5
SAGGGGPLGDNNQGADGVGNASGDWHCDSTWMGDR

SEQ ID NO: 2 - protein, 18 aa, Adeno-associated virus 5
SFVDHPPDWLEEVGEGLR

SEQ ID NO: 3 - protein, 5 aa, Adeno-associated virus 5
APTGK

SEQ ID NO: 4 - protein, 723 aa, Adeno-associated virus 5
SFVDHPPDWLEEVGEGLREFLGLEAGPPKPKPNQQHQDQARGLVLPGYNYLGPGNGLDRGEPVNRADEVAREHDISYNEQLEAGDNPYLKYNHADAEFQEKLADDTSFGGNLGKAVFQAKKRVLEPFGLVEEGAKTAPTGKRIDDHFPKRKKARTEEDSKPSTSSDAEAGPSGSQQLQIPAQPASSLGADTMSAGGGGPLGDNNQGADGVGNASGDWHCDSTWMGDRVVTKSTRTWVLPSYNNHQYREIKSGSVDGSNANAYFGYSTPWGYFDFNRFHSHWSPRDWQRLINNYWGFRPRSLRVKIFNIQVKEVTVQDSTTTIANNLTSTVQVFTDDDYQLPYVVGNGTEGCLPAFPPQVFTLPQYGYATLNRDNTENPTERSSFFCLEYFPSKMLRTGNNFEFTYNFEEVPFHSSFAPSQNLFKLANPLVDQYLYRFVSTNNTGGVQFNKNLAGRYANTYKNWFPGPMGRTQGWNLGSGVNRASVSAFATTNRMELEGASYQVPPQPNGMTNNLQGSNTYALENTMIFNSQPANPGTTATYLEGNMLITSESETQPVNRVAYNVGGQMATNNQSSTTAPATGTYNLQEIVPGSVWMERDVYLQGPIWAKIPETGAHFHPSPAMGGFGLKHPPPMMLIKNTPVPGNITSFSDVPVSSFITQYSTGQVTVEMEWELKKENSKRWNPEIQYTNNYNDPQFVDFAPDSTGEYRTTRPIGTRYLTRPL

(19) United States
(12) Patent Application Publication
(10) Pub. No.: US 2022/0268783 A1
(43) Pub. Date: Aug. 25, 2022

(54) METHODS FOR PEPTIDE MAPPING OF ADENO-ASSOCIATED VIRUS (AAV) PROTEINS

(71) Applicant: Waters Technologies Corporation, Milford, MA (US)

(72) Inventors: Ximo Zhang, Newton, MA (US); Stephan M. Koza, Lancaster, MA (US); Ying Qing Yu, Uxbridge, MA (US); Weibin Chen, Holliston, MA (US)

(73) Assignee: Waters Technologies Corporation, Milford, MA (US)

(21) Appl. No.: 17/314,452

(22) Filed: May 7, 2021

Related U.S. Application Data
(60) Provisional application No. 63/151,366, filed on Feb. 19, 2021.

(51) Int. Cl. G01N 33/68; G01N 30/20; G01N 30/22; G01N 30/72

(52) U.S. Cl. CPC G01N 33/6848; G01N 30/20; G01N 30/22; G01N 30/7233; G01N 2030/027

(57) ABSTRACT

The present disclosure relates to a method of characterizing proteins in a sample. The method includes: removing non-ionic surfactant from the sample via denaturing size-exclusion chromatography to form a denatured sample; eluting the denatured sample via liquid chromatography to collect fractions of the sample, wherein the fractions of the sample include a protein fraction; lyophilizing the protein fraction to increase protein concentration; reconstituting the lyophilized protein fraction with a buffer comprising a surfactant to denature the protein; digesting the denatured protein fraction with an enzyme; and analyzing the digested protein fraction.

### CROSS-REFERENCE TO RELATED APPLICATIONS

This application claims priority and benefit to U.S. Provisional Patent Application No. 63/151,366 filed on Feb. 19, 2021, entitled “Methods for Peptide Mapping of Adeno-Associated Virus (AAV) Proteins.” The content of which is incorporated herein by reference in its entirety.

### SEQUENCE LISTING

The instant application contains a Sequence Listing which has been submitted electronically in ASCII format and is hereby incorporated by reference in its entirety. Said ASCII copy, created on May 6, 2021, is named W-4332-US02_SL.txt and is 7,256 bytes in size

### FIELD OF THE TECHNOLOGY

The present disclosure relates generally to LC and LC/MS-based methods to deliver comprehensive characterization of proteins, such as adeno-associated viruses capsid proteins.

### BACKGROUND

Gene therapy refers to the modification or manipulation of gene expression or the genetic alteration of living cells for therapeutic purposes. Viral vectors, common for many gene therapies, have the primary functions of protecting the encapsulated genetic payload (RNA or DNA) and engaging in cellular targeting and trafficking. The most efficient viral vectors emerging from preclinical and clinical studies are adenovirus such as adeno-associated virus (AAV) and lentivirus. The most explored viral vector appears to be AAV, owing to its lower risk in humans and efficient transduction in a variety of cells and tissues.

AAV is a non-enveloped, single-stranded DNA parvovirus with many wild types found in nature. Structurally, AAV is an approximately 26-nm dimeter icosahedral capsid assembled from 60 viral protein (VP) monomers arranging into pentameric sub-structures. Each capsid contains three highly homologous VPs (VP1, VP2, and VP3) in a 1:1:10 proportion, where VP2 (˜65 kDa) is comprised of the entire amino acid sequence of VP3 (˜60 kDa) with an N-terminal extension, and VP1 (˜80 kDa) is an N-terminal extension of VP2. To date, at least 8 distinct serotypes of AAV have been used for gene therapy. While those AAV serotypes generally display 51-99% sequence homology, the differences in primary sequence of their VPs confer unique binding affinity toward various host cell receptors, leading to diverse tissue tropism.

### SUMMARY

Recombinant adeno-associated viruses (rAAVs) have emerged as the leading gene delivery platform due to their nonpathogenic nature and long-term gene expression capability. The AAV capsid, in addition to protecting the viral genome, plays an important role in viral infectivity and gene transduction, indicating the value of the constituent viral proteins (VPs) being well-characterized as part of gene therapy development. However, the limited sample availability and sequence homology shared by the VPs pose challenges to adapt existing analytical methods developed for conventional biologics.

The present disclosure discusses the development of RPLC/MS-based methods for characterization of proteins, such as AAV capsid proteins, at the peptide level with reduced sample consumptions. The present disclosure is generally directed to AAV capsid proteins. However, the present disclosure is also directed to all proteins. The methods are not to be construed as only applicable to AAV capsid proteins. The methods of the present disclosure allow the measurement of VP expression with fluorescence detection and intact mass/post-translational modifications (PTMs) analysis through a benchtop Time-of-Flight (ToF) mass spectrometer.

The general applicability and validity of the methods for gene therapy product development were demonstrated by applying the methods of the present disclosure to multiple common AAV serotypes. A one-hour enzymatic digestion method was also developed using 1.25 μg of AAV viral proteins, providing greater than 98% protein sequence coverage. The efficient and sensitive analyses of AAV capsid proteins enabled by the methods of the present disclosure can be used to improve the understanding and guide the development of AAV-related therapeutics throughout the commercialization process.

The continuous advancement and the expanding product pipelines of AAV-based gene therapeutics present challenges to product characterization. Similar to conventional biologics, well characterized AAVs are required to meet pre-determined specifications and regulatory standards for purity, potency, and safety. This industry demand calls for analytical technologies that are precise and accurate to monitor product quality and ensure batch-to-batch consistency. In addition, as more AAV therapeutics progressing from early discovery to clinical development, robustness, validity, and ease-of-use of the analytical methods become increasingly important to ensure the smooth transit into late stage development and commercialization.

One of the challenges in the analytical testing of rAAV vectors is the high degree of structural complexity. The multimeric nature as well as the variations of individual VPs make the structure of AAVs more complex than many monomeric recombinant protein therapeutics. To add further complication, the whole rAAV particle consists of not only proteins but also genetic materials. This clearly entails the development of methods beyond those applied to more established modalities to ensure that the unique nature of AAV biology is fully addressed.

The present disclosure describes a new digestion method that is compatible with low microgram quantities of AAVs was developed for peptide mapping. The protein loss was greatly reduced by minimizing buffer exchange and liquid transfer steps. Given the limited sample amount, the entire AAV capsid rather than the isolated VPs was selected to develop a single enzymatic digestion. To remove the surfactant from the AAVs, an 8-minute denaturing SEC-based method was developed to separate the AAV VPs from the surfactant.

In some aspects, the present disclosure provides a method of characterizing proteins in a sample. The method includes removing non-ionic surfactant from the sample via denaturing size-exclusion chromatography to form a denatured sample; eluting the denatured sample via liquid chromatography to collect fractions of the sample, wherein the fractions of the sample include a protein fraction; lyophilizing the protein fraction to increase protein concentration; reconstituting the lyophilized protein fraction with a buffer comprising a surfactant to denature the protein; digesting the denatured protein fraction with an enzyme; and analyzing the digested protein fraction.

In some embodiments, the method further includes adding methionine to the protein fraction, prior to lyophilizing the protein fraction.

In some embodiments, the protein fraction is less than 10 μg. In some embodiments, the protein fraction comprises adeno-associated virus capsid proteins.

In some embodiments, analyzing the digested protein fraction comprises analyzing with liquid chromatography-mass spectrometry. Analyzing the digested protein fraction via liquid chromatography-mass spectrometry can include analyzing intact mass/post-translational modifications of the digested protein fraction. In certain embodiments, analyzing the digested protein fraction via liquid chromatography-mass spectrometry can include a benchtop Time-of-Flight (ToF) mass spectrometer. In some embodiments, analyzing the digested protein fraction comprises measuring viral protein expression with fluorescence detection. And in some embodiments, analyzing the digested protein fraction comprises providing greater than 95% protein sequence coverage. Particularly, analyzing the digested protein fraction can comprise providing greater than 97% protein sequence coverage.

In some embodiments, the buffer further comprises a reducing agent. In certain embodiments, the buffer can further comprise a reducing agent and a metal chelator.

In some embodiments, the enzyme is trypsin.

In some embodiments, reconstituting the lyophilized protein fraction with a buffer comprising a surfactant to denature the protein is carried out at a temperature of greater than 65° C. Reconstituting the lyophilized protein fraction with a buffer comprising a surfactant to denature the protein can be carried out for less than about 5 minutes, for example, about 3 minutes.

In some embodiments, digesting the denatured protein fraction with an enzyme is carried out at a temperature ranging from about 30° C. to about 50° C. for about 50 minutes to about 70 minutes. In certain embodiments, digesting the denatured protein fraction with an enzyme is carried out for about 60 minutes.

### BRIEF DESCRIPTION OF THE DRAWINGS

The technology will be more fully understood from the following detailed description taken in conjunction with the accompanying drawings, in which:

FIG. 1 is a flowchart of an example of digestion workflow of the present disclosure.

FIG. 2A, FIG. 2B, FIG. 2C, and FIG. 2D display the removal of surfactant using denaturing size-exclusion chromatography (SEC).

FIG. 3A shows an XIC of AAV intact protein (VP3), which shows very little undigested proteins. FIG. 3B shows a total ion chromatogram (TIC) of an AAV peptide map.

FIG. 4A and FIG. 4B display peptide analysis of AAV5 VPs using approximately 1.25 μg proteins as the starting material in enzymatic digestion. FIG. 4B discloses SEQ ID NO: 4.

FIG. 5A, FIG. 5B, and FIG. 5C display identification of N-terminal peptides of AAV5 VPs via tandem mass spectrometry. FIGS. 5A-5C disclose SEQ ID NOS 1, 1-2, 2-3 and 3, respectively, in order of appearance.

### DETAILED DESCRIPTION

To characterize the rAAV vectors, X-ray crystallography and cryo-electron microscopy (cryo-EM) have been used to determine the three-dimensional (3D) structures of multiple AAV serotypes, showing only the VP3 common sequence is ordered. While unveiling critical structural information of the virions, these studies on 3D structures only provide a “snapshot” of the capsid topology in a low-energy state. Efforts have been put forth to perform molecular level studies that are typically undertaken throughout the development of conventional biotherapeutics. One of the focuses is to establish in-depth understanding of capsid composition (e.g., post-translations modifications (PTMs)) and their potential impact on viral infectivity and efficacy.

Gene therapy is a fast growing market. It delivers therapeutic genes to malfunctioned cells to cure the disease. AAV is the most popular vector/vehicle for gene delivery. The present disclosure discusses the LC-MS peptide mapping of adeno-associated virus (AAV) capsid proteins in gene therapy development. Some of the challenges in peptide mapping of AAV include: limited sample availability; non-ionic surfactant in formulation—difficult to remove and low protein recovery in buffer exchange; and low protein concentration. Typical sample preparation requires buffer exchange to remove surfactant and denaturant. The typical sample preparation also requires 10-20 μg protein, and the surfactant removal is often not effective. A smaller sample amount results in low protein recovery due to nonspecific adsorption on the filter. AAV is costly to produce and dosages are low (1E13 vg/mL sample contains ˜5 μg/mL VP1 & VP2 and ˜45 μg/mL VP3) (Zolgensma @2E13 and Luxturna @5E12). Low sample amounts may require MS detection for variant testing.

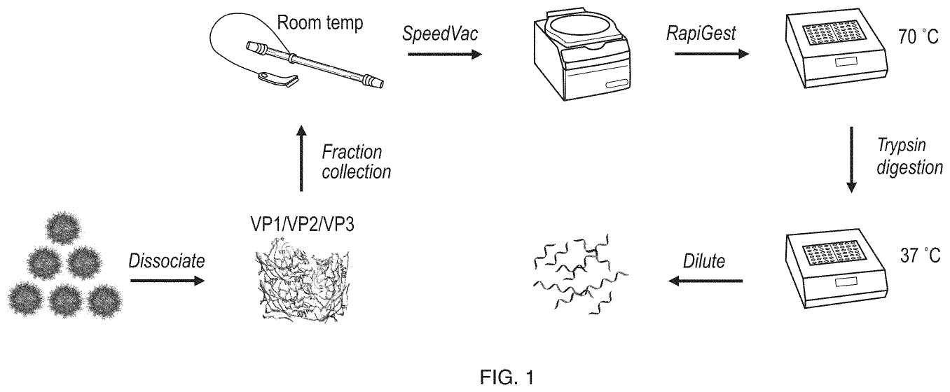

The present disclosure (see FIG. 1) discusses a solution that includes removing non-ionic surfactant using denaturing SEC, followed by protein lyophilization, RapiGest™ SF (available from Waters Corporation, Milford, Mass.) surfactant denaturation, and trypsin digestion.

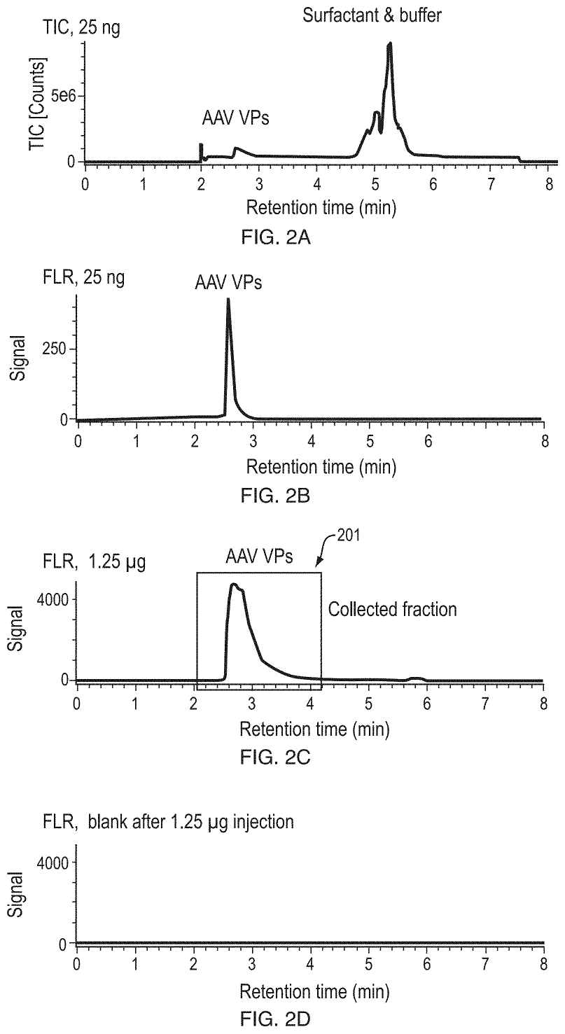

Denaturing SEC can remove surfactant with high recovery (see FIG. 2A, FIG. 2B, FIG. 2C, and FIG. 2D). Non-ionic surfactant can be removed by denaturing SEC (salt-free mobile phase). Protein load can be as low as 1.25 μg (the minimal sample amount to run on our LC/MS). Protein recovery is 98.6% based on the peak area of collected fraction and carryover.

In some examples, denaturing SEC can be completed by using a pressure-resistant sizing media housed within a pipette tip-based device, which interfaces with handheld pipettes or positive pressure sources. The volume of the sample will determine the volume of pressure-resistant sizing media being used. For example, a 10-50 μL sample requires a volume of 20-200 μL for pressure-resistant sizing media. Due to the low volume and quantity of samples, consumables with improved surface property that prevent nonspecific binding is important for high recovery (plasma treatment, QuanRecovery products available from Waters Corporation, Milford, Mass.).

AAV is costly to produce and dosages are low (1E13 vg/mL sample contains ˜5 μg/mL VP1 & VP2 and ˜45 μg/mL VP3) (Zolgensma @2E13 and Luxturna @5E12). Low sample amounts may require MS detection for variant testing. The method of the present disclosure (see FIG. 1) takes approximately 2.5 hours and solves the problems of other methods. Collected protein fraction was frozen and evaporated to dry to increase protein concentration. Methionine can be added to suppress method induced oxidation in some examples. MS-friendly reagent RapiGest™ SF surfactant (available from Waters Corporation, Milford, Mass.) eliminates the need of buffer exchange prior to digestion. Enzyme digestion can be carried out using MS grade, sequencing grade products in solution. Enzymes can also be immobilized on a solid support, which presents compatibility with RapiGest SF (available from Waters Corporation, Milford, Mass.).

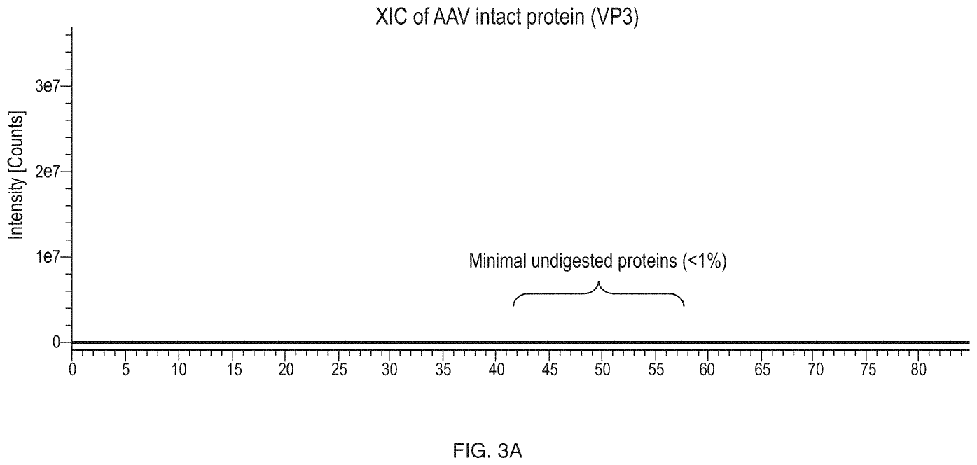

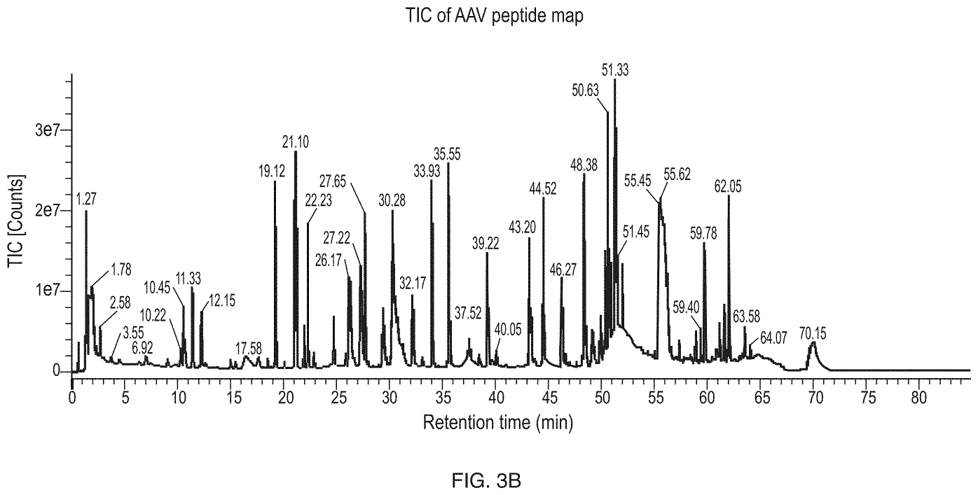

FIG. 3A shows an extracted ion chromatogram (XIC) of AAV intact protein (VP3), which shows very little undigested proteins. There were minimal undigested proteins (<1%). FIG. 3B shows a TIC of an AAV peptide map.

Establishing an in-depth understanding of capsid composition typically uses physicochemical methods such as mass spectrometry (MS) or liquid chromatography (LC) as they do not require product-specific analytical reagents such as monoclonal antibodies. Multiple analytical approaches have been applied including MS, isoelectric focusing, and cryo-EM, to characterize AAV8 samples produced by two manufacturing platforms, human HEK293 cells and Spodoptera frugiperda (Sf9) insect cells. These rAAV8 samples differed in PTMs, including glycosylation, acetylation, phosphorylation, and methylation, which shed lights on their observed potency differences in various target-cell types.

The present disclosure explores the mutational strategies to stabilize the amine groups and improve vector performance. To support AAV-based gene therapy development, one well-executed approach for peptides characterization is using reversed phase (RP) LC-MS, enabling the direct mass measurement and peptide sequence confirmation of 13 AAV serotypes for identification and purity assessment. Using a silica-hybrid based amide HILIC column and trifluoroacetic acid (TFA) as a mobile phase modifier, a separation can be performed on a wide range of AAV serotypes, and enhance the MS sensitivity by modifying the MS desolvation gas with propionic acid and isopropanol. While these methods are somewhat different, they do render insights into AAV sample quality and highlight the power of LC/MS as an effective analytical tool to facilitate the development of gene therapy products.

Besides the structural complexity, multiple challenges are still to be addressed for the analyses of rAAVs as compared to typical recombinant proteins. The difficulties in large-scale manufacturing and purification of AAVs result in low yield of samples. This can be problematic in supporting process development where often only micrograms of rAAVs are available for the analyst to cover a range of required assays. Another challenge is the low concentration of rAAV samples. As examples, Luxterna®, an ocular therapy, is formulated at 5×10¹² vector genomes per milliliter (vg/mL). This translates into protein concentrations of 30 μg/mL if 100% capsids contain transgene. With only 8% relative abundance, VP1 and VP2 are at low microgram levels in the formulated samples, increasing the risk of protein loss during sample preparation and analysis. As such, a sensitive and robust method that meets the challenge of structure complexity and sample scarcity of rAAV while delivering insightful information on product quality attributes is highly desirable.

The present disclosure explores the characterization of rAAV capsid using LC-MS techniques with an aim to develop robust, versatile, and sample-sparing methods that require minimal expertise to support the ever-growing activities in rAAV process development and manufacturing. These analyses were extended to encompass rAAV serotypes that show clinical promises and found broad applicability of the methods in measuring the critical quality attributes such as VP stoichiometry and the extent of PTMs (e.g., deamidation, oxidation) of capsid proteins. In general, the present technology utilizes SEC techniques to remove non-ionic surfactants and maintain the ability to have sufficient resolution to analyze the sample, especially for low concentrations and/or low quantity samples.

### EXAMPLES

### Enzymatic Digestion of AAV5 VPs

Prior to enzymatic digestion, denaturing size-exclusion chromatography (SEC) was used to remove a surfactant, like an ester such as polyoxyethylene sorbitol ester (e.g., Tween®20 available from MilliporeSigma, St. Louis, Mo.) polysorbate such as tween 20, from the AAV samples. Twenty-five (25.0) μL of AAV5 sample (1×10¹³ vg/mL) were injected onto a Acquity BEH SEC200 column (2.1×150 mm, 1.7 μm, 200 Å, Waters Corp, Milford, Mass.) maintained at 23° C. A mobile phase containing 0.1% trifluoroacetic acid, 0.1% formic acid, 10% acetonitrile, 20% isopropanol alcohol, and 69.8% water (all solvents and additives were from Fisher Scientific, Waltham, Mass.) was used at a flow rate of 0.08 mL/min.

The eluted AAV5 VPs were manually collected post-column from 2 to 4 minutes, to which 5 μL of 1 mM methionine solution was added and mixed in a 0.5-mL Protein Lobind® tube (Eppendorf, Hamburg, Germany). The mixture was immediately placed in a −80° C. freezer for rapid freezing, then lyophilized using a CentriVap vacuum concentrator (Labconco Corp., Kansas City, Mo.) within one hour. The dried AAV5 VPs were reconstituted in 5 μL of buffer solution consisting of 0.05% (w/v) RapiGest™ SF surfactant denaturant (Waters Corp., Milford, Mass.), 0.5 mM dithiothreitol (DTT, Fisher Scientific, Waltham, Mass.), 0.1 mM ethylenediaminetetraacetic acid (EDTA, Sigma-Aldrich, St. Louis, Mo.), and 50 mM pH8.0 Tris-HCl buffer (Fisher Scientific, Waltham, Mass.). The reconstituted AAV5 VPs was incubated at 70° C. for 3 minutes for denaturation. After cooling down to room temperature, the denatured AAV5 VPs solution was mixed with 2 μL of 0.1 μg/μL sequence grade modified trypsin (Promega, Madison, Wis.) and kept at 37° C. for 1 hour for proteolytic digestion. The digested AAV5 sample was then diluted using 18 μL of 10 mM methionine (in water) solution and placed in sample manager at 4° C. for LC-MS analysis.

### UHPLC-MS Peptide Mapping

The LC-MS analysis of AAV peptides was performed on the BioAccord System (Waters Corp, Milford, Mass.) with the same configuration as specified in the section of intact mass analysis. Twenty (20.0) μL (˜1 μg of proteins) of the tryptic digest of AAV VPs were injected onto an Acquity BEH C18 column (2.1×100 mm, 1.7 μm, 300 Å, Waters Corp, Milford, Mass.) maintained at 65° C. The peptides were separated using a mobile phase containing 0.1% LC-MS grade formic acid (Fisher Scientific, Waltham, Mass.) in water (A) and acetonitrile (B). At a flow rate of 0.2 mL/minute, the gradient was set as 1% B for 3 minutes, then ramped from 1% to 15% B in 18 minutes, 15-30% B in 48 minutes, 30-55% B in 51 minutes, 55-95% B in 65 minutes and maintained at 95% B until 67 minutes, and 1% B from 70 to 85 minutes for equilibration.

MS data was collected on the RDa detector under the “Full scan with fragmentation” mode. In this acquisition mode, both low-energy peptide precursor and the corresponding high-energy fragmentation data are acquired simultaneously. The other MS settings were as follows: capillary voltage, 1.2 kV; cone voltage, 20 V; fragmentation cone voltage, 60-120 V; desolvation temperature, 350° C.; scan range, 50-2000 m/z; and scan rate, 2 Hz. A SYNAPT-XS QuadrupoleTime-of-flight mass spectrometer (Waters Corp, Milford, Mass.) was also used for sequence confirmation with the following settings: capillary voltage, 2.2 kV; source temperature, 120° C.; collision energy, 20-50 eV; desolvation temperature, 350° C.; desolvation gas flow, 500 L/h; scan range, 50-2000 m/z; and scan rate, 2 Hz. Targeted MS/MS was used for the sequence confirmation of low abundance N-terminal peptides with the collision energy ramping at 30-50 eV. MS data were processed using the peptide mapping workflow within the waters_connect informatics platform. Mass tolerance was set as 10 ppm for precursor ions and 20 ppm for fragmentation ions. Up to one miss-cleavage with a minimum of 3 b-/y-ions matches were set as the criteria for peptide identification.

### Results and Discussion

### Enzymatic Digestion Method Development for AAV5 VPs

The present disclosure (see FIG. 1) discusses a solution that includes removing non-ionic surfactant using denaturing SEC, followed by protein lyophilization, RapiGest™ SF surfactant (available from Waters Corporation, Milford, Mass.) denaturation, and trypsin digestion.

Enzymatic digestion of proteins followed by LC/MS analysis of the proteolytic digest is commonly used for sequence confirmation and PTM identification of protein therapeutics. Although a full sequence coverage of VPs has been demonstrated in previous report with multi-enzyme digestions, the work used 10-20 μg AAV VPs to prepare the protein digest. Such sample requirement for a single peptide mapping workflow is difficult to satisfy due to the limited availability of AAV sample during early development phase. However, enzymatic digestion with greatly reduced AAV materials faces multiple challenges in sample preparation.

In addition to the restriction of low sample concentration, surfactants in AAV formulation buffers are problematic in MS analysis, such as poloxamer or tweens. While these surfactants were separated from intact VPs and did not cause problem in previous RPLC-MS analysis, they can severely interfere with LC-MS analysis of peptides. Typically, a buffer exchange step prior to digestion is needed to remove the surfactants along with enzyme inhibitors such as the denaturant and alkylation reagent. However, the complete removal of surfactants is challenging since their concentration are usually above the critical micelle concentration (CMC). Additionally, in common buffer exchange methods such as spin-filtering or dialysis, low protein concentration can lead to significant sample loss mostly due to the nonspecific adsorption to the membrane filter. Furthermore, in the case of low concentrated AAV VPs (˜50 μg/mL), the attempt to use less material in analysis will result in two difficult scenarios where either a very small volume of AAV sample is taken, or a larger volume sample with low protein concentration. From a sample preparation perspective, the small sample volume means that many common buffer exchange devices/protocols cannot be readily adopted. On the other hand, low concentrated protein samples would further decrease the enzymatic digestion rate and protein recovery. These challenges prevent the adoption of conventional enzymatic digestion protocols to the limited sample and call for a completely new sample preparation approach.

The present disclosure describes a new digestion method that is compatible with low microgram quantities of AAVs was developed for peptide mapping. The protein loss was greatly reduced by minimizing buffer exchange and liquid transfer steps. Given the limited sample amount, the entire AAV capsid rather than the isolated VPs was selected to develop a single enzymatic digestion. To remove the surfactant from the AAVs, an 8-minute denaturing SEC-based method was developed to separate the AAV VPs from the surfactant.

Using AAV5 as an example, 1.25 μg of VPs were injected and collected within a 2-minute window, resulting in a surfactant-free sample at a protein recovery over 95% (FIG. 2A-FIG. 2D). The fraction was lyophilized to dryness to remove organic solvents and increase the protein concentration for the following steps. Using 5-μL of reconstitution buffer that contains a MS-friendly denaturant, RapiGest™ SF surfactant (available from Waters Corporation, Milford, Mass.), at 0.05% (w/v), a one-pot denaturation and digestion method was developed. Although the AAV5 VPs do not have disulfide bonds in theory, a reducing reagent, DTT, was included in the buffer at low level to avoid disulfide pairing. This buffer composition can improve the solubility of the denatured proteins with minimal impact on enzymatic activities, making the buffer exchange step unnecessary prior to digestions. In addition, with the presence of the denaturant and reducing agent in the digestion buffer, alkylation was not required to prevent the reformation of disulfide bonds, which in turn eliminated the need for an additional buffer exchange.

FIG. 2A, 2B, 2C, and 2D display the removal of surfactant using denatured SEC. Using the developed 8-min method, AAV VPs were separated from the surfactant and other excipient as shown in FIG. 2A, the TIC of 25 ng AAVs. In FIG. 2B, under FLR detection, only the peak at 2.68 min were observed, confirming the peaks eluted after 4 min in FIG. 2A did not contain proteins. In FIG. 2C, the eluent of 1.25 μg AAVs was collected in the range of the rectangle 201 and used in the following enzymatic digestion, while minimal carryover was observed in FIG. 2D, which is a blank injection after fraction collection. The protein recovery was calculated to be 98.6% based on the area of the collected fraction over all peaks observed in FIG. 2C and FIG. 2D. Injection volume was 25 μL which can be adjusted based on the concentration of AAV samples.

The digestion method was further developed for trypsin-based proteolysis of AAV5 to minimize the sample preparation artifacts and digestion miscleavages. To reduce artifactual oxidation, 10 mM methionine was added as an oxygen scavenger to the collected AAV VPs from denatured SEC fractionation prior to lyophilization. This is an optional/result enhancing step that does not necessarily need to be performed in every instance. It was reported that the presence of DTT in the buffer can cause methionine oxidation due to the formation of hydrogen peroxide from metal-catalyzed reduction. Therefore, we added EDTA as a metal chelator and decreased the concentration of DTT in digestion solution to 0.5 mM. This DTT concentration is about 10-fold less than the concentration commonly used in the digestion methods for monoclonal antibodies. Despite of the lower concentration used in the current method, the molar ratio of DTT to the cysteine residues in AAV5 was still excessive (>25:1 ratio) to prevent the potential formation of disulfide bonds. The other digestion conditions were developed to achieve a balance between peptide miss-cleavages and method-induced modifications. To facilitate a more complete digestion, denaturation of AAV5 VPs was carried out at 70° C. prior to the tryptic digestion that was conducted at 37° C. with 1:10 enzyme to substrate ratio. To minimize sample preparation artifacts, e.g. oxidation and deamidation, the denaturation and digestion times were developed to be 3 minutes and 1 hour, respectively, resulting in a total sample preparation time of 2.5 hours.

This digestion method grants relatively flexible protein consumption. However, considering the low abundance of VP1 and VP2 on AAV capsid (<10%), the described 1.25 μg protein digest has generally precluded the use of UV detection, and is approaching the detection capability of the UHPLC/MS instrument configuration used for this study. While peptide mapping at analytical scale is preferred for robustness purposes, the sample usage in the digestion might be further decreased to meet the need of other analyses such as nanoLC/MS.

UHPLC/MS Analysis of AAV5 VPs Tryptic Digests

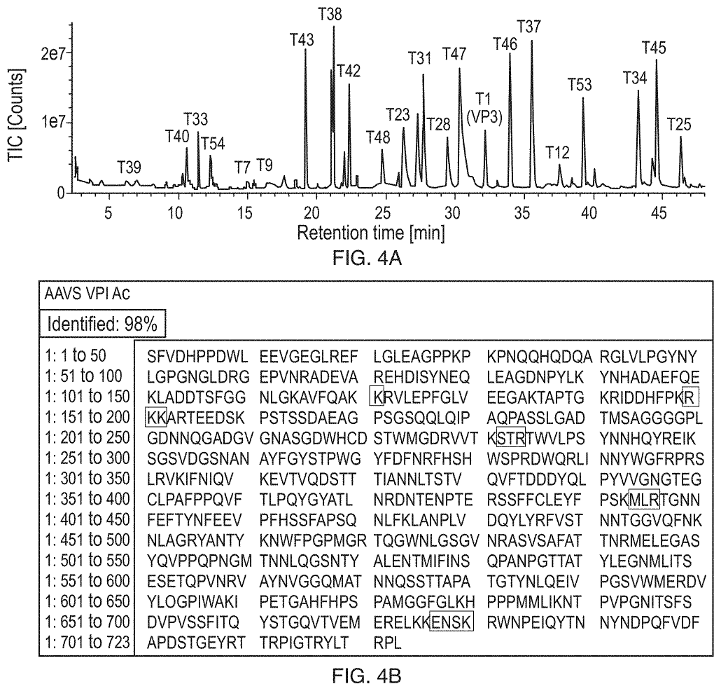

The digested AAV5 VPs were analyzed on the benchtop UHPLC-MS system to validate the developed digestion protocol for AAV protein sequence coverage. FIG. 4A and 4B display peptide analysis of AAV5 VPs using approximately 1.25 μg proteins as the starting material in enzymatic digestion. Data was processed in UNIFI peptide mapping workflow. Using a 45-minute gradient, the peptides were well separated on a C18 RP column with intensive MS signals shown in the TIC trace (FIG. 4A). The peptide identities were assigned based on observed masses and high-energy fragmentation ions. The coverage map of AAV5 VP1 (FIG. 4B) shows a 98% coverage of the protein sequence, and the peptides that were not identified are displayed in boxes (e.g., K, R, and MLR). The sequence coverages of VP2 and VP3 were both at 98% as well, as the only distinct peptide between their polypeptide backbones were those derived from their N-termini.

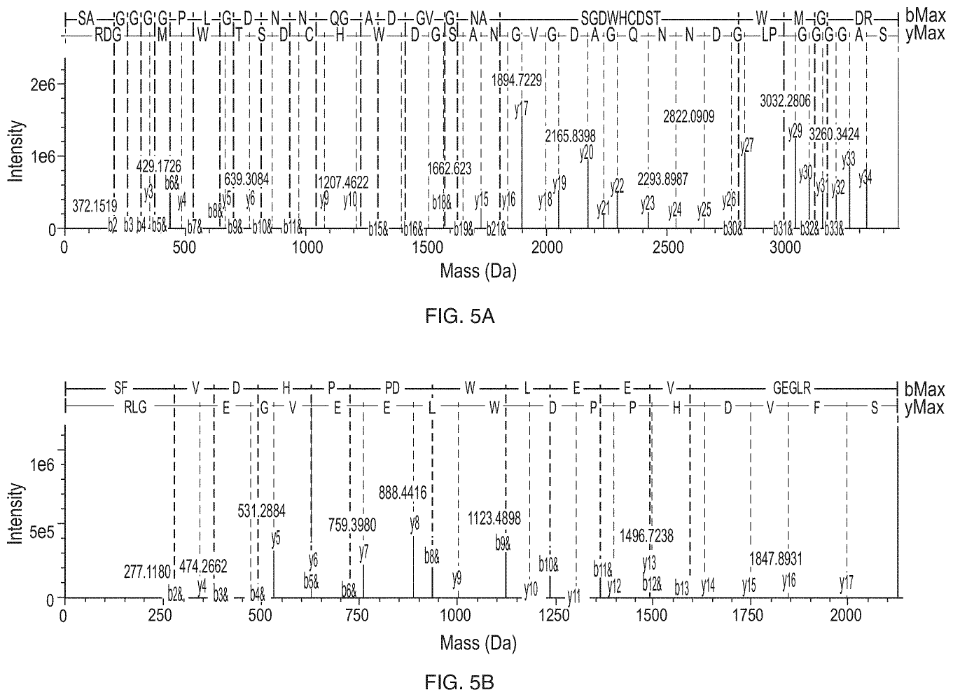

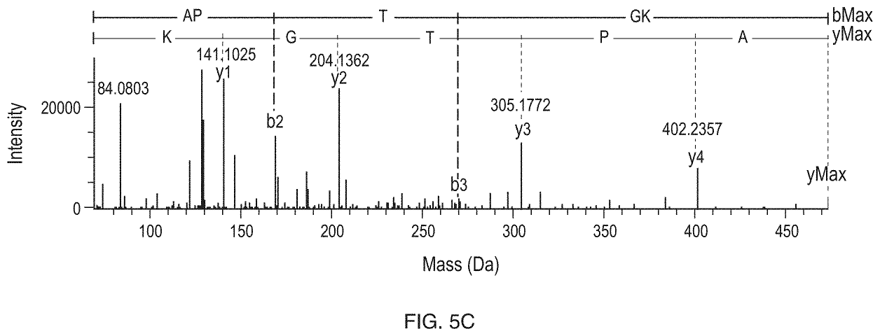

FIG. 5A, FIG. 5B, and FIG. 5C display identification of N-terminal peptides of AAV5 VPs via mass spectrometry. The identified primary ions were shown in the MS fragmentation spectra of (FIG. 5A) VP3 N-terminus (Ac)SAGGGGPLGDNNQGADGVGNASGDWHCDSTWMGDR (SEQ ID NO: 1), (FIG. 5B) VP1 N-terminus (Ac)SFVDHPPDWLEEVGEGLR (SEQ ID NO: 2), and (FIG. 5C) VP2 N-terminus APTGK (SEQ ID NO: 3). The fragmentation spectra of the singly charged VP2 N-terminus was obtained via target MS/MS at a higher collision energy. The deconvoluted MS fragmentation spectrum of VP3 N-terminus is shown in FIG. 5A with the annotated precursor mass (3460.3516 Da) and extensively distributed b-/y-ion series, confirming the peptide sequence of SAGGGGPLGDNNQGADGVGNASGDWHCDSTWMGDR (SEQ ID NO: 1). The MS data also showed a +42 Da mass shift compared to the theoretical mass of VP3 N-terminal peptide (mass accuracy of 0.2 ppm), suggesting the existence of N-acetylation associated with the peptide. The y₃₄ and yₘₐₓ ions with the 42 Da mass addition on the serine residue confirms the acetylation taking place at the N-terminus. Similarly, the VP1 N-terminal peptide, SFVDHPPDWLEEVGEGLR (SEQ ID NO: 2) (FIG. 5B) was identified with the mass accuracy of 2.2 ppm for the precursor, and N-acetylation was also found occurring on the serine residue. The VP2 N-terminal peptide, APTGK (SEQ ID NO: 3), consisting of only 5 amino acid residues, is only weakly retained on the RP column. In addition, this singly charged VP2 N-terminal peptide does not readily fragment under the general MS fragmentation settings used in the data independent acquisition (DIA) mode. Hence, target MS/MS data acquisition mode and a higher collision energy were employed to generate more extensive b- and y-ion fragments to confirm the peptide identity (FIG. 5C).

In this work, multiple LC and LC/MS-based methods were developed to deliver more comprehensive characterization of AAV capsid proteins. Analysis of several AAV serotypes demonstrated the general applicability of the method for routine use such as comparability studies. Using a combination of denaturing SEC fractionation and MS-friendly detergent, RapiGest™ SF surfactant (available from Waters Corporation, Milford, Mass.), the 1-hour enzymatic digestion method can generate high quality of tryptic digests with only 1.25 μg of AAV sample where nearly 100% sequence coverage is achieved for all VPs. In conclusion, the efficiency and sensitivity provided by these methods can benefit the analyses of AAV capsid proteins to improve the understanding and guide the design and manufacturing of new AAV therapeutics.

What is claimed is:

1. A method of characterizing proteins in a sample, the method comprising:
removing non-ionic surfactant from the sample via denaturing size-exclusion chromatography to form a denatured sample;
eluting the denatured sample via liquid chromatography to collect fractions of the sample, wherein the fractions of the sample include a protein fraction;
lyophilizing the protein fraction to increase protein concentration;
reconstituting the lyophilized protein fraction with a buffer comprising a surfactant to denature the protein;
digesting the denatured protein fraction with an enzyme; and
analyzing the digested protein fraction.

2. The method of claim 1, further comprising adding methionine to the protein fraction, prior to lyophilizing the protein fraction.

3. The method of claim 1, wherein the protein fraction is less than 10 μg.

4. The method of claim 1, wherein the protein fraction comprises adeno-associated virus capsid proteins.

5. The method of claim 1, wherein analyzing the digested protein fraction comprises analyzing with liquid chromatography-mass spectrometry.

6. The method of claim 5, wherein analyzing the digested protein fraction via liquid chromatography-mass spectrometry comprises analyzing intact mass/post-translational modifications of the digested protein fraction.

7. The method of claim 5, wherein analyzing the digested protein fraction via liquid chromatography-mass spectrometry comprises a benchtop Time-of-Flight (ToF) mass spectrometer.

8. The method of claim 1, wherein analyzing the digested protein fraction comprises measuring viral protein expression with fluorescence detection.

9. The method of claim 1, wherein analyzing the digested protein fraction comprises providing greater than 95% protein sequence coverage.

10. The method of claim 1, wherein analyzing the digested protein fraction comprises providing greater than 97% protein sequence coverage.

11. The method of claim 1, wherein the buffer further comprises a reducing agent.

12. The method of claim 1, wherein the buffer further comprises a reducing agent and a metal chelator.

13. The method of claim 1, wherein the enzyme is trypsin.

14. The method of claim 1, wherein reconstituting the lyophilized protein fraction with a buffer comprising a surfactant to denature the protein is carried out at a temperature of greater than 65° C.

15. The method of claim 1, wherein reconstituting the lyophilized protein fraction with a buffer comprising a surfactant to denature the protein is carried out for less than about 5 minutes.

16. The method of claim 15, wherein reconstituting the lyophilized protein fraction with a buffer comprising a surfactant to denature the protein is carried out for about 3 minutes.

17. The method of claim 1, wherein digesting the denatured protein fraction with an enzyme is carried out at a temperature ranging from about 30° C. to about 50° C.

18. The method of claim 1, wherein digesting the denatured protein fraction with an enzyme is carried out for about 50 minutes to about 70 minutes.

19. The method of claim 18, wherein digesting the denatured protein fraction with an enzyme is carried out for about 60 minutes.
